(19) United States
(12) Patent Application Publication
(10) Pub. No.: US 2001/0041174 A1
(43) Pub. Date: Nov. 15, 2001

(54) USE OF IMPLANTED ENCAPSULATED CELLS EXPRESSING GLUTAMATE TRANSPORTER PROTEINS FOR THE TREATMENT OF NEURODEGENERATIVE DISEASES

(76) Inventor: NAJAM SHARIF, ARLINGTON, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/443,765

(22) Filed: Nov. 19, 1999

Related U.S. Application Data
(60) Provisional application No. 60/109,866, filed on Nov. 24, 1998.

(51) Int. Cl. A61K 48/00; C07H 21/04; C12P 21/02

(52) U.S. Cl. 424/93.21; 435/328; 435/320.1; 435/69.1; 530/350; 536/23.5

(57) ABSTRACT

Encapsulated cell devices useful for the treatment of ophthalmic, brain and spinal cord diseases and disorders are disclosed. The encapsulated cell devices contain cells expressing glutamate transporter proteins.

The present application claims priority to U.S. provisional Ser. No. 60/109,866 filed Nov. 14, 1998.

The present invention relates to encapsulated cells expressing glutamate transporters and methods of use for the treatment of glaucoma, macular degeneration other retinal diseases and other degenerative diseases of the brain and spinal cord.

### BACKGROUND OF THE INVENTION

The excitatory amino acid glutamate (“GLUT”) is an endogenous chemical which acts as a potent neurotransmitter when it is released from certain nerves cells (neurons) in many organs and tissues of the body including the brain, spinal cord and retina (Sharif, Multiple synaptic receptors for neuroactive amino acid transmitters—new vistas, Int. Rev. Neurobiol., volume 26, pages 85-150 (1985)). GLUT is released in the synaptic cleft (space between the nerve terminal and neighboring nerve and glial cells). GLUT stimulates the post-synaptic neurons via specific receptors to produce biological functions such as tissue contraction, hormone release or transmission of visual messages. Following its neurotransmitter action, GLUT is rapidly and actively transported into the nerve terminal. GLUT uptake is also effected by the numerous glial cells surrounding the neurons via glial cell-specific transport mechanisms.

Uptake of GLUT is necessary in order maintain normal extracellular GLUT levels in the synaptic space. An excess of GLUT causes an over stimulation of the neurons, resulting in cytotoxicity. Neuronal cell death leads to such diseases as glaucoma, optic neuritis (Dryer et al., Elevated glutamate levels in the vitreous body of humans and monkeys with glaucoma, Arch. Ophthalmology, volume 114, pages 299-305 (1996); Chew and Ritch, Neuroprotection: the next breakthrough in glaucoma? Proceedings of the third annual optic nerve rescue and restoration think tank and related retinal diseases, J. Glaucoma, volume 6, pages 263-266, (1997)), and Alzheimer's, Lou Gehrig's and Huntington's diseases (Choi, Excitotoxic cell death, J. Neurobiology, volume 23, pages 1261-1276, (1992)).

Such excitotoxic (neurotoxic) effects of GLUT have been studied extensively and have now been linked to the retinal and brain disorders mentioned above. For example, it has been shown that exogenous administration of GLUT in the vitreous of animal eyes (Sisk et al., Histological changes in the inner retina of albino rats following intravitreal injection of monosodium L-glutamate, Graef's Arch. Clin. Exp. Ophthalmol., volume 223, pages 250-258 (1985)), or in vitro application to retinal cells (Reif-Lehrer et al., Effects of monosodium L-glutamate on chick embryo retina in culture, Invest. Ophthalmol. Vis. Sci., volume 14, pages 114-124 (1975)), causes significant retinal damage. Consequently, the effective removal/uptake of excessive GLUT seems to be crucial for neuronal cell viability.

Aberrant synaptic GLUT accumulation may result from a number of different biological conditions. For example, GLUT accumulation may occur as a result of ischemic/hypoxic and/or hypoglycemic conditions, GLUT transport mechanism defects, GLUT metabolism defects, or in certain disease processes (e.g., status epilepticus and stroke) wherein excessive stimulation of the nerve terminals leads to excessive release of GLUT.

One of the major functions of the glial cells surrounding nerve terminals and neurons in the retina, brain and spinal cord is to remove, recycle/detoxify the synaptic GLUT, thereby preventing its accumulation in the synapse. Thus, if the active transport (i.e., uptake) of GLUT by the nerve terminal and/or glial cells is compromised, GLUT accumulates in the space around nerve cells leading them to be literally “excited to death” (Choi, Excitotoxic cell death, J. Neurobiology, volume 23, pages 1261-1276 (1992)).

Failure or loss of the GLUT transport system may seriously aggravate such neurotoxic damage. Even minor amounts of extracellular GLUT may be sufficient to induce excitotoxic death in brain cells (Frandsen and Schousboe, Development of excitatory amino acid induced cytotoxicity in cultured neurons, Int. J. Dev. Neurosci., volume 8, pages 209-216 (1990)). Moreover, under conditions of energy failure (e.g. ischemia and/or hypoglycemia), GLUT-transporters (primarily on glial cells) may work in reverse, leading to a further increases in GLUT levels in the extracellular space around nerve cells and, consequently, an increased potential for nerve cell death and pathological consequences (Gegelashvili and Schousboe, High affinity glutamate transporters: regulation of expression and activity, Mol. Pharmacol., volume 52, pages 6-15, (1997)).

The GLUT-transport mechanism is an energy-dependent process and is mediated by specific transporter proteins present on the glutamatergic nerve terminals and glial cells. Glial cells in the central nervous tissues are known as astrocytes, and in the retina these cells are known as Muller cells. As stated above, the glial cells take-up and recycle GLUT, thereby preventing a build-up of GLUT. To date, at least five specific human GLUT-transporter proteins have been cloned, namely GLAST, GLT1, EAAC1, EAAT4 and ASCT/SATT (Gegelashvili and Schousboe, High affinity glutamate transporters: regulation of expression and activity, Mol. Pharmacol., volume 52, pages 6-15, (1997)). The loss or absence of GLT1 has been linked to Lou Gehrig's disease (ALS) (Bristol and Rothstein, Glutamate transporter gene expression in amyotrophic lateral sclerosis motor cortex, Ann. Neurol., volume 39, pages 676-679, (1996)). The loss or absence of other GLUT transporters on astrocytes and neurons has also been shown to lead to elevated extracellular GLUT levels, excitotoxic neurodegeneration, paralysis and epileptic seizures (Rothstein et al., Knockout of glutamate transporters reveals a major role for astroglial transport in excitotoxicity and clearance of glutamate, Neuron, volume 16, pages 675-686 (1996)).

GLUT levels are significantly elevated in the vitreous of the posterior chamber of the human eye in patients with glaucoma and in monkeys with laser-induced glaucoma (Dryer et al., Elevated glutamate levels in the vitreous body of humans and monkeys with glaucoma, Arch. Ophthalmol., volume 114, pages 299-305, (1996)). Ischemic-borne retinopathies and optic neuropathies, including glaucoma, are multifactorial diseases (i.e., diseases wherein a number of factors play a role in the etiology of the disease) which lead to retinal and optic nerve damage and ultimately blindness. While it is not known whether there is a loss of or defect in the GLUT transport system in the human retina of glaucoma patients, the elevated vitreal GLUT levels may be deleterious and may cause severe retinal dysfunction if not reduced in a timely manner. Likewise, in brain and spinal diseases, an excess of GLUT appears to play a role in the pathology (e.g., patients with chronic epilepsy, ALS and the other diseases mentioned above). The rapid removal of excess GLUT in these tissues would be expected to provide significant benefit to the patient.

The use of encapsulated normal or bioengineered cells to secrete biologically active compounds have been previously described. (See, e.g., Aebischer et al., Infrathecal delivery of CNTF using encapsulated genetically modified xenogeneic cells in amyotrophic lateral sclerosis patients, Nature Medicine, volume 2, pages 696-699 (1996); Linder, et al., Implanatation of encapsulated cathecholamine and GDNF-producing cells in rats with unilateral dopamine depletions and Parkinsonian Symptoms, Expt. Neurol., volume 132, pages 62-67 (1995) and WIPO Publication No. WO 95/05452)). In addition, other publications describe methods of implanting encapsulated cells in a host (WO 95/28166); use of certain graft polymers for use in the cell encapsulation procedures (WO 94/25503), and the use of microporous capsules useful as implantation devices (WO 94/10950). WO 96/02646A2 discloses a method for controlling growth of cells which are encapsulated in a bioartificial organ (BAO). The publication further discloses a method whereby cells are proliferated in vitro and a balance between proliferation and differentiation is controlled when the cells are encapsulated in a BAO.

U.S. Pat. No. 5,158,881 discloses a method and system for encapsulating cells within a semi-permeable polymeric membrane by co-extruding an aqueous cell suspension and a polymeric solution through a common port to form a tubular extrudate having a polymeric outer coating which encapsulates the cell suspension. U.S. Pat. No. 5,283,187 discloses a method for encapsulating living cells within a polymeric membrane by co-extruding an aqueous cell suspension and polymeric solution through a common port having at least one concentric bore to form a tubular extrudate having a polymeric membrane which encapsulates the cell suspension. WO 95/01203 discloses an apparatus and method for sealing implantable hollow fiber encapsulation devices.

However, none of the patents or publications above disclose methods for the removal of detrimental substances from the body, or particularly GLUT, using implanted, encapsulated living cells.

### SUMMARY OF THE INVENTION

The present invention is directed to devices and methods of use for the treatment of ophthalmic, brain and spinal cord diseases and disorders which are characterized by excessive extracellular GLUT. More specifically, the present invention is directed to devices which encapsulate cells expressing GLUT transporter proteins. The GLUT transporter cells are derived from normal human tissues and cultured in vitro, or are bioengineered to express GLUT transporter proteins.

The methods of the present invention involve the implantation of the encapsulated cell devices in the body at or near sites of pathology involving GLUT excitotoxicity. The present invention methods are particularly directed to the treatment of glaucoma, macular degeneration, ischemic-borne retinopathies, optic neuritis, diabetic neuropathies and other retinal degenerative diseases. The methods of the present invention are also directed to the treatment of brain and spinal cord diseases or conditions where an excess of GLUT is involved in the disease, including, but not limited to, stroke, epilepsy and related seizure disorders, and Alzheimer's, Huntington's and Lou Gehrig's diseases.

### DETAILED DESCRIPTION OF THE INVENTION

The present invention is directed to biocompatible devices which encapsulate cells expressing GLUT transporter proteins, and methods of use. The term “biocompatible” is used herein to refer collectively to both the intact macrocapsule and the cells it contains. Specifically, “biocompatible” refers to the ability of the implanted intact macrocapsule and its contents to avoid detrimental effects on the body's various protective systems, such as the induction of an immune response, the induction of foreign body fibrotic responses, or other undesirable effects such as systemic or cytotoxic effects. The encapsulated cell devices are selectively permeable. As used herein, “selectively permeable” refers to encapsulated cell devices that are permeable to GLUT, cellular nutrients and waste products but not permeable to the encapsulated cells, immune defense systems of the host or potential viruses shed from the host or encapsulated cells. The most important aspect of the devices of the present invention is that they are permeable to GLUT. Extracellular GLUT permeates through the capsule and is absorbed by the encapsulated cells, thereby effectively removing the toxic, excess GLUT from the extracellular environment of the neurons.

The encapsulated cells are glial cells (Muller and/or astrocytes) which contain constitutive GLUT transporter proteins, or other suitable cells which have been bioengineered to express one or more of the human GLUT transporters proteins. The appropriate number of these cells are then suitably encapsulated in suitable devices by methods described herein, and then implanted in the patient at or near the tissue to be treated, e.g., the vitreal space in the patient's eye to treat glaucoma or other retinal degenerative diseases known to be caused by elevated GLUT levels, the inter- or intra-cerebral space to treat brain diseases, or the interor intra-spinal space to treat spinal cord diseases. The devices and methods of the present invention employ a sufficient amount of cells such that the aggregate number of GLUT transporters expressed is in an amount effective to reduce the amount of extracellular GLUT in the region of the host where the device is implanted. As used herein, such a GLUT transporter amount is referred to as “an effective amount of GLUT transporters.”

The specific isolation and in vitro culture of human neurons, glial and other types of living cells useful in the present invention, are well known and practiced in the art (see, e.g., Jacoby and Pastan, Methods in Enzymology: Cell culture, Academic Press (1979); Freshney, Culture of animal cells, 3rd Edition, John Wiley and Sons, (1994)). Thus, cells of these types which normally and constitutively express GLUT transporter proteins can be readily isolated in reasonable yields from human cadaver tissues for encapsulation and implantation in human patients, according to the present invention. The use of non-human cells expressing GLUT transporter proteins is also contemplated by the present invention.

The cells of the present invention may also be modified to exhibit prolonged life spans. There are numerous methods for bioengineering living human or non-human cells to immortalize them such that they continue to maintain certain cellular levels suitable for the specific function. (See e.g., Coca-Prados et al. Transformation of human ciliary epithelial cells by simian virus 40: induction of cell proliferation and retention of β-adrenergic receptors, Proc. Nat. Acad. Sci. USA, volume 83, pages 8754-8758 (1986); Aizawa et al., Establishment of a variety of human bone marrow stromal cell lines by the recombinant SV40-adenvirus vector, J. Cell Physiol., volume 148, pages 245-251 (1991); Pfeifer et al., Simian virus 40 large tumour-immortalized normal human liver epithelial cells express hepatocyte characteristics and metabolize chemical carcinogens, Proc. Nat. Acad. Sci USA, volume 90, pages 5123-5127 (1993); Sharif et al., Immortalized human corneal epithelial cells for ocular inflammation and toxicity studies, Invest. Ophthalmol. Vis. Sci., (Suppl), volume 38, no. 3349 (1997)).

The cells useful in the present invention may also be derived from cells which originally did not express GLUT transporter proteins. There are methods known to those skilled in the art to specifically make cells of different types express certain proteins on their cell surface (e.g., Feigner et al., Lipofection: a highly efficient, lipid-mediated, DNA-transfection procedure, Proc. Natl. Acad. Sci. USA, volume 84, pages 7413-7417 (1987)) such as GLUT transporters (Shafqat et al., Cloning and expression of a novel Na⁺-dependent neutral amino acid transporter structurally related to mammalian Na⁺/glutamate cotransporters, J. Biol. Chem., volume 268, pages 15351-15355 (1993); Arriza et al., Functional comparisons of three glutamate transporter subtypes cloned from human motor cortex, J. Neurosci., volume 14, pages 4449-5569 (1994); Inoue et al., Cloning and expression of a bovine glutamate transporter, Mol. Brain Res., volume 28, pages 343-348 (1995)). Thus, bioengineering methods are well known to those skilled in the art and may be employed in the present invention for the preparation of cells useful in the devices contemplated herein.

As stated above, cells may be transformed to express GLUT transporter proteins by incorporation of GLUT transporter genes. Genes encoding GLUT transporter proteins have been cloned and their nucleotide sequences have been published. (See, e.g., Gegelashvili and Schousboe, High affinity glutamate transporters: regulation of expression and activity, Mol. Pharmacol., volume 52, pages 6-15, (1997)). U.S. Pat. No. 5,739,284 (Hediger, et al.) and U.S. Pat. No. 5,225,323 (Lams) also describe GLUT transporter DNA sequences and methods of use, and are incorporated herein by reference. Other sources of GLUT gene sequences are publicly available from depositories such as the GenBank (Basel, Switzerland). Genes encoding the GLUT transporter proteins useful in the present invention which are not publicly available may be obtained using standard recombinant DNA methods such as PCR amplification, genomic and cDNA library screening with oligonucleotide probes. (See, e.g., U.S. Pat. No. 5,049,493 (Khosla et al.), U.S. Pat. No. 5,082,670 (Gage et al.) U.S. Pat. No. 5,169,762 (Gray, et al.); the contents of the foregoing patents are incorporated herein by reference.) Accordingly, any of the genes described above, or those yet to be elucidated, which code for GLUT transporter proteins may be employed in bioengineered cells of the present invention.

Among the genes particularly useful in this invention are those genes cloned and described in Gegelashvili and Schousboe, High affinity glutamate transporters: regulation of expression and activity, Mol. Pharmacol., volume 52, pages 6-15, (1997).

A gene encoding a GLUT transporter protein can be inserted into a cloning site of a suitable expression vector by using standard techniques. It will be appreciated that more than one gene may be inserted into a suitable expression vector for insertion into living cells. These techniques are well known to those skilled in the art. The expression vector containing the gene of interest may then be used to transfect the cell line to be used in the methods of this invention. Standard transfection techniques such as calcium phosphate coprecipitation, DEAE-dextran transfection or electroporation may be utilized. Commercially available mammalian transfection kits may be purchased from various sources (e.g., Stratagene, LaJolla, Calif.).

A wide variety of host/expression vector combinations may be used to express the gene encoding the biologically active molecule of interest. Long-term, stable in vivo expression is achieved using expression vectors (i.e., recombinant DNA molecules) in which the gene encoding the biologically active molecule is operatively linked to a promoter that is not subject to down regulation upon implantation in vivo in a mammalian host. Suitable promoters include, for example, the early and late promoters of SV40 or adenovirus and other known non-retroviral promoters capable of controlling gene expression. Useful expression vectors, for example, may consist of segments of chromosomal, non-chromosomal and synthetic DNA sequences, such as various known derivatives of SV40 and known bacterial plasmids, e.g., plasmids from E. coli including, pBR322, pCR1, pMB9, pUC and their derivatives.

Expression vectors containing drug resistance sequences may also be employed in the transfection methods contemplated herein. For example, geneticin (G418) or hygromycin drug selection genes may be incorporated in the expression vectors used herein. Such vectors successfully incorporated in the host may then be selected based on their resistance to the respective drug.

A variety of different mammalian promoters may be employed to direct the expression of the genes for the GLUT transporter protein genes. Various promoters and their employment are well known in the art.

The cell types that can be employed for encapsulated cell therapy within the scope of this invention include cells from allogeneic and xenogeneic sources. One of the principal advantages of our encapsulated approach rests with the immunoisolatory properties of the membranes of this invention, and their ability to support cells that otherwise would not be appropriate for transplantation (i.e., non-human sources, immortalized and/or tumor cell lines). A particular advantage to using xenogeneic over allogeneic cells is that in the unlikely event of membrane failure, the xenogeneic cells are more likely to be targeted for destruction by the immune system when compared to allogeneic cells. Furthermore, xenogeneic sources are easy to obtain and their use precludes the necessity for the use of human tissue which is difficult to obtain and fraught with societal and ethical considerations. In addition, human tissue may contain adventitious agents that are more readily transmitted to the transplantation recipient. Finally, use of xenogeneic tissue and cell lines for transplantation in humans removes the risks associated with the handling and processing of human tissue.

The cell lines transformed according to this invention are capable of providing long-term, stable expression of GLUT transporter proteins. Such long-term, stable expression can be achieved by increasing or amplifying the copy number of the transgene encoding the GLUT transporter protein(s), using amplification methods well known in the art. Such amplification methods include, e.g., DHFR amplification (see, e.g., U.S. Pat. No. 4,740,461 (Kaufman et al.), the contents of which are incorporated herein by reference).

A variety of biocompatible immunoisolatory capsules are suitable for delivery of molecules according to this invention. Such capsules will allow for the passage of metabolites, nutrients and therapeutic substances while minimizing the detrimental effects of the host immune system.

Useful biocompatible polymer capsules or housings, comprise (1) a core which contains a cell or cells, preferably either suspended in a liquid medium or immobilized within a hydrogel or extracellular matrix, and (2) an encapsulating, biocompatible, selectively permeable, matrix or membrane (jacket) which is sufficient to protect isolated cells from immunological factors.

The core of the polymer capsule is constructed to provide a suitable local environment for the continued viability and function of the cells isolated therein.

Many transformed cells or cell lines are most advantageously isolated within a capsule having a liquid core. For example, cells can be isolated within a capsule whose core comprises a nutrient medium, optionally containing a liquid source of additional factors to sustain cell viability and function, such as those present in fetal bovine or equine serum.

Suitably, the core may be composed of a matrix formed by a hydrogel which stabilizes the position of the cells in cell clumps. The term “hydrogel” herein refers to a three dimensional network of cross-linked hydrophilic polymers. The network is in the form of a gel, substantially composed of water. Examples of hydrogels include collagen, laminin, alginate, celluloses, chondroitin sulfate, hyaluronic acid, and other cross-linked polymers such as polyvinyl alcohol, xanthan, and guar gums, and pharmaceutically acceptable salts thereof.

As stated above, the encapsulated cell devices are immunoisolatory. The thickness of the capsule can vary, but it will always be sufficiently thick to prevent direct contact between the encapsulated cells and the extra-capsular space. The thickness of the capsular wall will generally range between about 5 and 200 microns. While the thickness of the wall may vary depending on the particular devices employed, i.e., the types and quantity of cells contained therein, the geometry of the device and particular capsular materials employed, a thickness of from about 10 to 100 microns is preferred.

Various commercially available polymers and polymer blends can be used to manufacture the capsule jacket, including polyacrylates (including acrylic copolymers), polyvinylidenes, polyvinyl chloride copolymers, polyurethanes, polystyrenes, polyamides, cellulose acetates, cellulose nitrates, polysulfones, polyvinylidines polyphosphazenes, polyacrylonitriles, poly(acrylonitrile/covinyl chloride), as well as derivatives, copolymers and mixtures thereof.

The capsule can be any configuration appropriate for maintaining biological activity and providing access for the uptake of GLUT, including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient's blood is vessels, are not preferred. Certain shapes, such as rectangles, patches, disks, cylinders, and flat sheets offer greater structural integrity and are preferable where retrieval is desired.

The capsules must provide, in at least one dimension, sufficiently close proximity of any isolated cells in the core to the surrounding tissues of the recipient in order to maintain the viability and function of the isolated cells. In general, the capsules will have a volume of about 1 to 100 μL.

Any of the encapsulated cell devices described above may be used in the devices and methods of the present invention. The following U.S. patents and WIPO publications further describe useful cell encapsulated devices, and are incorporated herein to the extent they disclose useful devices and bioengineering techniques useful in the preparation of the devices of the present invention: WO 95/05452; WO 96/02646A2; WO 94/25503; WO 94/10950; U.S. Pat. Nos. 5,158,881; 5,283,187; WO 95/01203.

The manufacture of spherical or tubular capsules has been disclosed in previous patents (e.g., U.S. Pat. No. 5,283,187). Tubular capsules are preferred because fine tubes can be coiled, twisted or otherwise deposited in various shapes to provide configurations which are appropriate for implantation sites in the specific part(s) of the human eye, brain ventricle(s) or spinal cord cerebrospinal fluid space. Furthermore, the tubular structure allows easy removal from the host when necessary. A detailed description of tubular capsule construction is disclosed in U.S. Pat. No. 5,283,187, which is the preferred device of the present invention.

Various surgical techniques are available for the intravitreal implantation of a device of the present invention. For example, U.S. Pat. No. 5,378,475 (Smith et al.) discloses methods for the surgical implantation of intravitreal devices. The contents of the foregoing reference to the extent it discloses methods for the surgical implantation of intravitreal devices is incorporated herein.

The methods of the present invention are also useful in the treatment of brain disorders caused by high GLUT levels. The treatment of brain diseases involves the implantation of devices of the present invention in the closest brain ventricle or other suitable site(s) close to the area(s) with anticipated, elevated GLUT concentration.

The methods of the present invention are also useful in the treatment of spinal cord disorders (e.g. ALS). The treatment of spinal cord diseases involves the implantation of the devices of the present invention in such a manner (e.g. intrathecally) to be accessible to the cerebrospinal fluid which most likely would have the high GLUT levels.

I claim:

1. An encapsulated cell device comprising an enclosed capsule having an exterior and an interior, wherein at least a portion of the capsule is permeable such that glutamate and general cellular nutrients and waste products may ingress and egress between the interior and exterior of the capsule, and wherein the interior comprises cells which express an effective amount of GLUT transporter proteins.

2. A device according to claim 1, wherein the cells are normal human glial cells or bioengineered cells expressing GLUT transporter proteins.

3. A device according to claim 1, wherein the cells are bioengineered to express one or more GLUT transporter proteins.

4. A device according to claim 1, wherein the capsule comprises one or more permeable and/or non-permeable polymer(s) selected from the group consisting of: polyacrylates (including acrylic copolymers), polyvinylidenes, polyvinyl chloride copolymers, polyurethanes, polystyrenes, polyamides, cellulose acetates, cellulose nitrates, polysulfones, polyvinylidines polyphosphazenes, polyacrylonitriles, poly(acrylonitrile/covinyl chloride), as well as derivatives, copolymers and mixtures thereof.

5. A method for treating humans with neurodegenerative diseases characterized by excessive, extracellular GLUT levels, which comprises implanting an encapsulated cell device at or near the neural tissue at risk, said device comprising an enclosed capsule having an exterior and an interior, wherein at least a portion of the capsule is permeable such that glutamate and general cellular nutrients and waste products may ingress and egress between the interior and exterior of the capsule, and wherein the interior comprises cells which express an effective amount of GLUT transporter proteins.

6. A method according to claim 5, wherein the cells are normal human glial cells or bioengineered cells expressing GLUT transporter proteins.

7. A method according to claim 5, wherein the cells are bioengineered to express GLUT transporter proteins.

8. A method according to claim 5, wherein the capsule comprises one or more permeable and/or non-permeable polymer(s) selected from the group consisting of: polyacrylates (including acrylic copolymers), polyvinylidenes, polyvinyl chloride copolymers, polyurethanes, polystyrenes, polyamides, cellulose acetates, cellulose nitrates, polysulfones, polyvinylidines polyphosphazenes, polyacrylonitriles, poly(acrylonitrile/covinyl chloride), as well as derivatives, copolymers and mixtures thereof.

9. A method for treating ophthalmic diseases which comprises implanting a cell encapsulated device at least partially exposed to the vitreous of an eye, said device comprising an enclosed capsule having an exterior and an interior, wherein at least a portion of the capsule is permeable such that glutamate and general cellular nutrients and waste products may ingress and egress between the interior and exterior of the capsule, and wherein the interior comprises cells which express an effective amount of GLUT transporter proteins.

10. A method according to claim 9, wherein the cells are normal human glial cells or bioengineered cells expressing GLUT transporter proteins.

11. A method according to claim 9, wherein the cells are bioengineered to express GLUT transporter proteins.

12. A method according to claim 9, wherein the capsule comprises one or more permeable and/or non-permeable polymer(s) selected from the group consisting of: polyacrylates (including acrylic copolymers), polyvinylidenes, polyvinyl chloride copolymers, polyurethanes, polystyrenes, polyamides, cellulose acetates, cellulose nitrates, polysulfones, polyvinylidines polyphosphazenes, polyacrylonitriles, poly(acrylonitrile/covinyl chloride), as well as derivatives, copolymers and mixtures thereof.

13. A method according to claim 9, wherein the ophthalmic disease is glaucoma, macular degeneration, an ischemic-borne retinopathy, a diabetic neuropathy, optic neuritis or other degenerative retinal diseases or conditions wherein extracellular GLUT is elevated above normal levels.

14. A method for treating brain or spinal cord diseases which comprises implanting a cell encapsulated device at or near the diseased cerebral or spinal tissue, said device comprising an enclosed capsule having an exterior and an interior, wherein at least a portion of the capsule is permeable such that glutamate, general cellular nutrients and waste products may ingress and egress between the interior and exterior of the capsule, and wherein the interior comprises cells which express an effective amount of GLUT transporter proteins.

15. A method according to claim 14, wherein the cells are normal human glial cells or bioengineered cells expressing GLUT transporter proteins.

16. A method according to claim 14, wherein the cells are bioengineered to express IS GLUT transporter proteins.

17. A method according to claim 14, wherein the capsule comprises one or more permeable and/or non-permeable polymer(s) selected from the group consisting of: polyacrylates (including acrylic copolymers), polyvinylidenes, polyvinyl chloride copolymers, polyurethanes, polystyrenes, polyamides, cellulose acetates, cellulose nitrates, polysulfones, polyvinylidines polyphosphazenes, polyacrylonitriles, poly(acrylonitrile/covinyl chloride), as well as derivatives, copolymers and mixtures thereof.

18. A method according to claim 14, wherein the disease is stroke, head trauma, epilepsy or other seizure disorders, Alzheimer's disease, Huntington's diseases, Lou Gehrig's disease (ALS) or other diseases or conditions wherein extracellular GLUT is elevated above normal levels.
